(19) United States
(12) Patent Application Publication
(10) Pub. No.: US 2024/0018207 A1
(43) Pub. Date: Jan. 18, 2024

(54) INTERFERON TAU FC-FUSION PROTEINS AND METHODS FOR TREATING CORONAVIRUS INFECTIONS

(71) Applicant: Southlake Pharmaceuticals, Inc., Cambridge, MA (US)

(72) Inventors: Yuhua George Zhang, Auburndale, MA (US); Wendy Wanjin Tang, College Station, TX (US)

(21) Appl. No.: 18/027,675

(22) PCT Filed: Sep. 22, 2021
(86) PCT No.: PCT/US21/51591

§ 371 Date: Mar. 22, 2023

Related U.S. Application Data
(60) Provisional application No. 63/083,077, filed on Sep. 24, 2020.

(51) Int. Cl. C07K 14/555; C12N 15/63; A61P 31/14

(52) U.S. Cl. CPC C07K 14/555; C07K 2319/30; A61P 31/14; C12N 15/63

(57) ABSTRACT

The invention provides novel Fc-fusion proteins of interferon tau and compositions thereof, methods of their preparation and therapeutic use thereof in treating coronavirus (e.g., COVID-19 virus/SARS-CoV-2 and hCoV229E) viral infections, and related diseases and conditions.

### PRIORITY CLAIMS AND RELATED PATENT APPLICATIONS

This application claims the benefit of priority to U.S. Provisional Application Ser. No. 63/083,077, filed Sep. 24, 2020, the entire content of which is incorporated herein by reference.

### TECHNICAL FIELD OF THE INVENTION

The invention generally relates to novel biologics and therapeutic uses thereof. More particularly, the invention provides novel interferon tau Fc-fusion proteins, compositions thereof, and methods of their preparation and therapeutic use in treating coronavirus (e.g., COVID-19 virus/SARS-CoV-2 and hCoV229E) viral infections, and related diseases and conditions.

### BACKGROUND OF THE INVENTION

The recent COVID-19 outbreak, caused by severe acute respiratory syndrome coronavirus 2 (SARS-CoV-2), has severely compromised the health and daily life of the general public and hampered the economic growth of this country and the rest of the world. In addition to the vaccines, there is strong demand for an effective therapeutic for treating infected patients.

Members of coronavirus family, of which COVID-19 is a member, are positive-sense single-stranded RNA virus genomes in the size ranging from 26 to 32 kilobases. They are enveloped and non-segmented. They have the largest known viral RNA genome. The virion has a nucleocapsid, which consists of genomic RNA and phosphorylated nucleocapsid (N) protein. N protein is contained inside phospholipid bilayers and wrapped by two different types of spike proteins: the spike glycoprotein trimmer (S) possessed by all CoVs, and the hemagglutinin-esterase (HE) that is present in a few CoVs. There are also membrane (M) protein (a type III transmembrane glycoprotein) and the envelope (E) protein next to the S proteins in the virus envelope. (Li, et al. 2020) J Med Virol 92(4): 424-432.)

There are four genera in the coronavirus family Coronaviridae, i.e., α, β, γ, and δ coronaviruses. 30 CoVs are found to infect humans, mammals, fowl, and other animals. α—and β—CoVs cause human infections. CoVs are common human pathogens. Human Coronavirus 229E (hCoV-229E) is an α-CoV responsible for common cold. SARS (severe acute respiratory syndrome CoV) related viruses (including COVID-19 virus/SARS-CoV-2) and MERS (Middle East respiratory syndrome CoV) related viruses, and another common cold virus OC43 are β-CoVs. They all belong to the same coronavirus family Coronavirividae. These viruses cause severe pneumonia, dyspnea, renal insufficiency, and even death possibly due to over-reacted immune response. (Li, et al. 2020 J Med Virol 92(4): 424-432; Chan, et al. 2015 Clin Microbiol Rev 28(2): 465-522; Cheng, et al. 2007 Clin Microbiol Rev 20(4): 660-694; Zumla, et al. 2016 Nat Rev Drug Discov 15(5): 327-347.)

Various antiviral agents are currently under investigation for treating the viral infection; however, none have yet been scientifically established to be effective in clearing the virus or mitigating mortality in published randomized controlled trials. As of now, Remdesivir is the only agent that may have an effect on the time it takes to recover from infection by the virus. Remdesivir was authorized by US FDA to treat patients hospitalized with severe COVID-19. Several other agents, such as Hydroxychloroquine or Chloroquine, which were previously thought or proclaimed to be effective, have since been shown to have little effect or may even be harmful. Containment and mitigation strategies so far have had limited impact in slowing down the spread of the highly contageous and fast-moving COVID-19 virus. (Sanders, et al. 2020 JAMA 323(18):1824-1836; Mahase, 2020 British Med J 369: m1798; Mahase, 2020 British Med J 370: m3049; Aschenbrenner, 2020 Am JNurs 120(7): 26.). Despite vaccinations and Remdesivir usage, the daily COVID19 new and death cases in US are soaring (https://coronavirus.jhu.edα/covid-19-daily-video).

Thus, safe and effective treatments are urgently needed in the fight against the COVID-19 pandemic.

### BRIEF DESCRIPTION OF THE DRAWINGS

FIG. 1A. Exemplary anti-SARS-CoV-2 activity of IFNT and IFNT Fc-fusion proteins. U619ZFC020-5 is IFNT with His and FLAG tags; U619ZFC020-7 is IFNT with C-terminal IgG1 Fc fusion; U619ZFC020-13 is IFNT with N-terminal IgG1 Fc fusion.

FIG. 1B. Exemplary cell viability assay of IFNT and Fc-IFNT fusion proteins.

FIG. 1C. Exemplary anti-SARS-CoV-2 activity of the reference compounds: remdesivir, chloroquine, hydroxychloroquine, aloxistatin, calpain inhibitor IV.

FIG. 2. Exemplary dose-response curves of IFNT and IFNT Fc-fusion proteins, Remdesivir in inhibiting hCoV 229E in CPE and cell viability assay.

FIG. 3A-C. Exemplary of cloning strategies of IFNT-His-Flag and IFNT Fc-fusion proteins.

FIG. 4A-C. Exemplary SDS-PAGE and Western Blot analysis of IFNT-His-Flag and IFNT Fc-fusion proteins.

FIG. 5 Protein sequences of original IFNT.

FIG. 6A-C. Exemplary plasmid maps of IFNT-His-Flag and IFNT Fc-fusion proteins.

### SUMMARY OF THE INVENTION

The invention is based in part on the unexpected discovery of novel therapeutic compositions and treatment methods based on interferon tau (IFNT), or IFNT Fc-fusion proteins comprising IFNT and a for treating or reducing coronavirus infections, in particular COVID-19 infections, and influenza/common cold infections. The compositions and methods of the invention are also useful in treating and reducing diseases and conditions related to coronavirus infections, in particular COVID-19 infections, such as pneumonia, acute respiratory distress syndrome (ARDS), inflammations and cardiovascular disorders, and as well as common cold and flu.

In one aspect, the invention generally relates to a fusion protein that comprises IFNT, or a fragment thereof and the Fc portion of a human IgG comprising a hinge region, an IgG CH2 domain and an IgG CH3 domain.

In another aspect, the invention generally relates to an isolated fusion protein disclosed herein.

In yet another aspect, the invention generally relates to a purified fusion protein disclosed herein.

In yet another aspect, the invention generally relates to an isolated nucleic acid encoding a fusion protein disclosed herein.

In yet another aspect, the invention generally relates to an expression vector comprising the nucleic acid encoding a fusion protein disclosed herein.

In yet another aspect, the invention generally relates to a host cell comprising the expression vector comprising the nucleic acid encoding a fusion protein disclosed herein.

In yet another aspect, the invention generally relates to a composition comprising a fusion protein disclosed herein.

In yet another aspect, the invention generally relates to a pharmaceutical composition comprising a therapeutically effective amount of a fusion protein disclosed herein.

In yet another aspect, the invention generally relates to a unit dosage form comprising a fusion protein disclosed herein.

In yet another aspect, the invention generally relates to a unit dosage form comprising a pharmaceutical composition disclosed herein.

In yet another aspect, the invention generally relates to a method for preparing the fusion protein, the method comprises: culturing a host cell comprising an expression vector comprising a nucleic acid encoding the fusion protein disclosed herein; expressing the nucleic acid to fusion protein; and recovering the fusion protein from the host cell culture.

In yet another aspect, the invention generally relates to a method for treating or reducing a coronavirus infection, or a related disease or condition, comprising administering to a subject in need thereof a therapeutically effective amount of a fusion protein disclosed herein, and a pharmaceutically acceptable excipient, carrier, or diluent.

In yet another aspect, the invention generally relates to a method for inhibiting viral replication in cells, comprising administering to a subject in need thereof a therapeutically effective amount of a fusion protein comprising IFNT, or a fragment thereof, and the Fc portion of a human IgG comprising a hinge region, an IgG CH2 domain and an IgG CH3 domain, and a pharmaceutically acceptable excipient, carrier, or diluent.

In yet another aspect, the invention generally relates to use of a fusion protein comprising IFNT, or a fragment thereof, and the Fc portion of a human IgG comprising a hinge region, an IgG CH2 domain and an IgG CH3 domain, for treating a coronavirus infection, or a related disease or condition.

In yet another aspect, the invention generally relates to use of a fusion protein comprising IFNT, or a fragment thereof, and the Fc portion of a human IgG comprising a hinge region, an IgG CH2 domain and an IgG CH3 domain, in preparation of a medicament for treating a coronavirus infection, or a related disease or condition.

### Definitions

Unless defined otherwise, all technical and scientific terms used herein have the same meaning as commonly understood by one of ordinary skill in the art to which this invention belongs. The following terms, unless indicated otherwise according to the context wherein the terms are found, are intended to have the following meanings.

As used herein, the term “cell” refers to any prokaryotic, eukaryotic, primary cell or immortalized cell line, any group of such cells as in, a tissue or an organ. Preferably the cells are of mammalian (e.g., human) origin and can be infected by one or more pathogens.

As used herein, the terms “disease” or “disorder” refer to a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the terms “Fc” or “Fc region”, refer to the constant region of a full-length immunoglobulin excluding the first constant region immunoglobulin domain. For IgG, Fc comprises immunoglobulin domains CH2, CH3 and the lower hinge region between CH1 and CH2.

As used herein, the term “host cell” refers to an individual cell or a cell culture that can be or has been a recipient of any recombinant vector(s) or isolated polynucleotide(s). A host cell can be a transfected, transformed, transduced or infected cell of any origin, including prokaryotic, eukaryotic, mammalian, avian, insect, plant or bacteria cells, or it can be a cells of any origin that can be used to propagate a nucleic acid described herein. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell that comprises a recombinant vector of the invention may be called a “recombinant host cell.”

Most cells include, without limitation, the cells of mammals, plants, insects, fungi and bacteria. Bacterial cells include, without limitation, the cells of Gram-positive bacteria such as species of the genus Bacillus, Streptomyces and Staphylococcus and cells of Gram-negative bacteria such as cells of the genus Escherichia and Pseudomonas. Fungal cells include, preferably, yeast cells such as Saccharomyces, Pichia pastoris and Hansenula polymorpha. Insect cells include, without limitation, cells of Drosophila and Sf9 cells. Plant cells include, among others, cells from crop plants such as cereals, medicinal or ornamental plants or bulbs. Suitable mammal cells for the present invention include epithelial cell lines (porcine, etc.), osteosarcoma cell lines (human, etc.), neuroblastoma cell lines (human, etc.), epithelial carcinomas (human, etc.), glial cells (murine, etc.), liver cell lines (monkey, etc.). CHO cells (Chinese Hamster Ovary), COS cells, BHK cells, cells HeLa, 911, AT1080, A549, 293 or PER.C6, human ECCs NTERA-2 cells, D3 cells of the line of mESCs, human embryonic stem cells such as HS293 and BGVO1, SHEF1, SHEF2 and HS181, cells NIH3T3, 293T, REH and MCF-7 and hMSCs cells.

As used herein, the term “high dosage” is meant at least 5% (e.g., at least 10%, 20%, 50%, 100%, 200%, or even 300%) more than the highest standard recommended dosage of a particular compound for treatment of any human disease or condition.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., of a IL15 or IL15Ra sequence), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A preferred example of algorithm that is suitable for determining percent sequence identity and sequence similarity are the BLAST algorithms, which are described in Altschul et al. 1977 Nuc. Acids Res. 25:3389-3402 and Altschul et al. 1990 J Mol. Biol. 215:403-410, respectively. BLAST software is publicly available through the National Center for Biotechnology Information on the worldwide web at ncbi.nlm.nih.gov/. Both default parameters or other non-default parameters can be used. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

As used herein, the terms “IgG” or “Immunoglobulin G” refer the main type of antibody found in blood and extracellular fluid. IgG is a polypeptide belonging to the class of antibodies that are substantially encoded by a recognized immunoglobulin gamma gene. In humans, IgG comprises the subclasses or isotypes IgG1, IgG2, IgG3, and IgG4.

As used herein, the term “inhibit” refers to any measurable reduction of biological activity. Thus, as used herein, “inhibit” or “inhibition” may be referred to as a percentage of a normal level of activity.

As used herein, the term an “isolated” molecule (such as a polypeptide or polynucleotide) is one that has been manipulated to exist in a higher concentration than in nature or has been removed from its native environment. For example, a subject antibody is isolated, purified, substantially isolated, or substantially purified when at least 10%, or 20%, or 40%, or 50%, or 70%, or 90% of non-subject-antibody materials with which it is associated in nature have been removed. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “polynucleotide,” “nucleic acid molecule,” “nucleotide,” “oligonucleotide,” and “nucleic acid” are used interchangeably herein to refer to polymeric forms of nucleotides, including ribonucleotides as well as deoxyribonucleotides, of any length. They can include both double-, single-stranded or triple helical sequences and include, but are not limited to, cDNA from viral, prokaryotic, and eukaryotic sources; mRNA; genomic DNA sequences from viral (e.g., DNA viruses and retroviruses) or prokaryotic sources; RNAi; cRNA; antisense molecules; recombinant polynucleotides; ribozymes; and synthetic DNA sequences. The term also captures sequences that include any of the known base analogs of DNA and RNA. Nucleotides can be referred to by their commonly accepted single-letter codes.

Polynucleotides are not limited to polynucleotides as they appear in nature, and also include polynucleotides where unnatural nucleotide analogues and inter-nucleotide bonds appear. A nucleic acid molecule may comprise modified nucleic acid molecules (e.g., modified bases, sugars, and/or internucleotide linkers). Non-limitative examples of this type of unnatural structures include polynucleotides wherein the sugar is different from ribose, polynucleotides wherein the phosphodiester bonds 3′-5′ and 2′-5′ appear, polynucleotides wherein inverted bonds (3′-3′ and 5′-5′) appear and branched structures. Also, the polynucleotides of the invention include unnatural inter-nucleotide bonds such as peptide nucleic acids (PNA), locked nucleic acids (LNA), C1-C4 alkylphosphonate bonds of the methylphosphonate, phosphoramidate, C1-C6 alkylphosphotriester, phosphorothioate and phosphorodithioate type. In any case, the polynucleotides of the invention maintain the capacity to hybridize with target nucleic acids in a similar way to natural polynucleotides.

Unless otherwise indicated or obvious from context, a particular nucleic acid sequence also implicitly encompasses conservatively modified variants thereof (e.g., degenerate codon substitutions) and complementary sequences, as well as the sequence explicitly indicated. Degenerate codon substitutions can be achieved by generating sequences in which the third position of one or more selected (or all) codons is substituted with mixed-base and/or deoxyinosine residues. (Batzer et al. 1991 Nucleic Acid Res. 19:5081; Ohtsuka et al. 1985 J. Biol. Chem. 260:2605-2608; Rossolini et al. 1994 Mol. Cell. Probes 8:91-98.)

As used herein, the terms “protein” and “polypeptide” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, a polypeptide may refer to a protein which includes modifications, such as deletions, additions, and substitutions (generally conservative in nature), to the native sequence, as long as the protein maintains the desired activity. These modifications may be deliberate or may be accidental. Amino acids can be referred to herein by either their commonly known three letter symbols or by the one-letter symbols recommended by the IUPAC-IUB Biochemical Nomenclature Commission.

As used herein, the term “purified” refers to a protein that may be substantially or essentially free of components that normally accompany or interact with the protein as found in its naturally occurring environment, i.e. a native cell, or host cell in the case of a recombinantly produced protein. A protein that may be substantially free of cellular material includes preparations of protein having less than about 30%, less than about 20%, less than about 15%, less than about 10%, less than about 5%, less than about 4%, less than about 3%, less than about 2%, or less than about 1% (by dry weight) of contaminating protein(s). When a protein or variant thereof is recombinantly produced by the host cells, the protein may be present at about 30%, at about 20%, about 15%, about 10%, about 5%, about 4%, about 3%, about 2%, or about 1% or less of the dry weight of the cells. When a protein or variant thereof is recombinantly produced by the host cells, the protein may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. Thus, a “substantially purified” protein may have a purity level of at least about 80%, specifically, a purity level of at least about 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Proteins and prodrugs of the present invention are, subsequent to their preparation, preferably isolated and/or purified to obtain a composition containing an amount by weight equal to or greater than 80% (“substantially pure”), which is then used or formulated as described herein. In certain embodiments, the compounds of the present invention are more than 95% pure.

As used herein, the term “recombinant,” with respect to a nucleic acid molecule, means a polynucleotide of genomic, cDNA, viral, semisynthetic, and/or synthetic origin which, by virtue of its origin or manipulation, is not associated with all or a portion of the polynucleotide with which it is associated in nature. The term “recombinant”, as used with respect to a protein or polypeptide, means a polypeptide produced by expression of a recombinant polynucleotide. The term “recombinant” as used with respect to a host cell means a host cell into which a recombinant polynucleotide has been introduced.

As used herein, the term “recombinant virus” refers to a virus that is genetically modified by the hand of man. The phrase covers any virus known in the art.

As used herein, the term “sample” refers to a sample from a human, animal, or to a research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

As used herein, the terms “subject” and “patient” are used interchangeably herein to refer to a living animal (human or non-human). The subject may be a mammal. The terms “mammal” or “mammalian” refer to any animal within the taxonomic classification mammalia. A mammal may be a human or a non-human mammal, for example, dogs, cats, pigs, cows, sheep, goats, horses, rats, and mice. The term “subject” does not preclude individuals that are entirely normal with respect to a disease or condition, or normal in all respects.

As used herein, the term “therapeutically effective amount” refers to the dose of a therapeutic agent or agents sufficient to achieve the intended therapeutic effect with minimal or no undesirable side effects. A therapeutically effective amount can be readily determined by a skilled physician, e.g., by first administering a low dose of the pharmacological agent(s) and then incrementally increasing the dose until the desired therapeutic effect is achieved with minimal or no undesirable side effects.

As used herein, the terms “treatment” or “treating” a disease or disorder refers to a method of reducing, delaying or ameliorating such a condition, or one or more symptoms of such disease or condition, before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “at least” a specific value is understood to be that value and all values greater than that value.

As used herein, “more than one” is understood as 2, 3, 4, 5, 6, 7, 8, 9, 10, 11, 12, 13, 14, 15, 16, 17, 18, 19, 20, 25, 30, 40, 50, 100, etc., or any value therebetween.

Unless specifically stated or obvious from context, as used herein, the term “or” is understood to be inclusive.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Any compositions or methods disclosed herein can be combined with one or more of any of the other compositions and methods provided herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### DETAILED DESCRIPTION OF THE INVENTION

The invention provides novel therapeutic compositions and treatment methods, which considerably expand the therapeutic options for coronavirus infections, in particular COVID-19 infections, and related diseases and conditions. The disclosed invention is also useful in treating and reducing diseases and conditions related to coronavirus infections, in particular COVID-19 infections, such as pneumonia, ARDS, inflammation and cardiovascular disorders, and as well as common cold or flu.

Interferons (IFNs) were initially discovered as proteins able to cause an antiviral condition in cells. IFNs are small proteins or glycoproteins secreted by eukaryotic cells to fight against viral infection and other antigenic stimuli. There are three classes of IFNs according to their different chemical, immunological, and biological properties: interferon I, II and III. All type I IFNs bind to the cell surface IFN-α/β receptor (IFNAR). IFNT is a member of type-I interferon (IFN) family. Within type I IFN family, it is most similar to IFN omega (IFNW) with about 70% amino acid (AA) identity. It has about 50% of AA identity with IFN-α (IFNA) and about 25% AA identity with IFN-β (IFNB). Unlike IFNA, IFNB, and other Type I interferons, a striking feature of IFNT is that it does not have cytotoxicity even at high concentrations. (Soos et al. 1995 J Immunol 155(5): 2747-2753.) Ovine IFNT binds to type I IFN receptors on cells with high affinity, but less strongly than IFNA and IFNB, to induce comparable antiproliferative, antiviral and immunomodulatory activities, but without the known cytotoxicity of IFNA and IFNB. (Pontzer et al. 1991 Cancer Res 51(19): 5304-5307; Soos et al. 1995 J Immunol 155(5): 2747-2753; Bazer et al. 2010 Mol Hum Reprod 16(3): 135-152.)

IFNT is the pregnancy recognition signal secreted from trophectoderm of ruminant (cow, sheep, and goat) conceptuses (embryo and associated membranes). There is no functionally active human analog of IFNT. Ovine IFNT has been shown to have antiviral, anti-proliferative and immunomodulatory effects. (Bazer et al. 2010 Mol Hum Reprod 16(3): 135-152.)

Severe toxicity was observed in animal studies as well as in clinical trials with IFNA and IFNB, including tachycardia, nausea, weight loss, leucopenia, and neutropenia. (Degr6 1974 Int J Cancer. 14(6): 699-703; Fent et al. 1987 Trends Pharmacol Sci 8:100-105). In contrast, in vitro, in vivo and in human studies with IFNT have shown minimal toxicity. For example, in a head-to-head comparison, significant levels of toxicity were detected in mice fed with IFNA or IFNB, but no lymphocyte depression in mice fed with IFNT or PBS was observed. Oral- and IV— administered IFNT have significantly lower toxicity than IFNA and IFNB. (See, e.g., U.S. Pat. No. 6,372,206 Bi; WO 2005/087255A2; Waubant et al. 2007 Rev Neurol (Paris) 163(6-7):688-96.)

Another unique advantage of IFNT is its oral availability. Oral administration of IFNT increases energy metabolism, reduces adiposity, and alleviates adipocytes inflammation and insulin resistance in rats and mice. (Tekwe et al. 2013 Biofactors 39(5): 552-563; Ying et al. 2014 PLoS One 9(6): e98835.) Human clinical studies have shown that thrice daily oral doses of 3 mg of IFNT for up to nine months was safe and well tolerated.

As first discovered by the present inventors and disclosed herein. IFNT exhibits remarkable antiviral activity against coronaviruses, in particular, against COVID-19 virus SARS-CoV-2.

Fc-fusion proteins are proteins with an N-terminal or C-terminal Fc domains of IgG. Fc-fusion proteins have dimeric structures. The dimers are cross-connected by a pair of disulfide bonds between cysteines in alongside subunits. Among various types of IgG antibodies, IgG1 has the highest affinity for Fc receptors. (Hogg 1988 Immunol Today, 9: 185-7; Levin, et al. 2015 Trends Biotechnol, 33: 27-34; Woof, et al. 1984 Mol Immunol, 21: 523-7.)

IFNT Fc-fusion proteins disclosed herein exhibit distinctive beneficial biological properties, including for example, extended half-life and enhanced immunogenicity.

In one aspect, the invention generally relates to a fusion protein that comprises IFNT, or a fragment thereof and the Fc portion of a human IgG comprising a hinge region, an IgG CH2 domain and an IgG CH3 domain.

In certain embodiments, the human IgG is IgG1.

In certain embodiments, the Fc portion of the human IgG is at the C terminal of IFNT.

In certain embodiments, the Fc portion of the human IgG is at the N terminal of IFNT.

In certain embodiments, the IFNT comprises a mammalian IFNT.

In certain embodiments, the IFNT comprises a non-human mammalian IFNT.

In certain embodiments, the IFNT comprises recombinant IFNT.

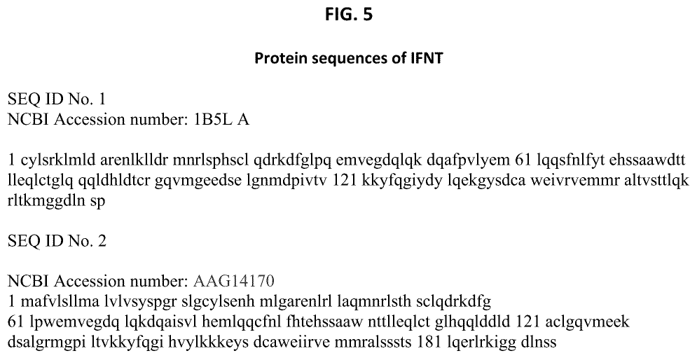

In certain embodiments, the fusion protein comprises an amino acid sequence that is at least 80% homologous with SEQ ID No. 1 or SEQ ID No. 2. In certain embodiments, the fusion protein comprises an amino acid sequence having at least 80% homologous with SEQ ID NO. 1. In certain embodiments, the fusion protein comprises an amino acid sequence having at least 80% homologous with SEQ ID NO. 2. (FIG. 5)

In another aspect, the invention generally relates to an isolated fusion protein disclosed herein.

In yet another aspect, the invention generally relates to a purified fusion protein disclosed herein.

In yet another aspect, the invention generally relates to an isolated nucleic acid encoding a fusion protein disclosed herein.

In yet another aspect, the invention generally relates to an expression vector comprising the nucleic acid encoding a fusion protein disclosed herein.

In certain embodiments, the expression vector is an adenovirus, an adeno-associated virus, or gene therapy virus vectors.

In yet another aspect, the invention generally relates to a host cell comprising the expression vector comprising the nucleic acid encoding a fusion protein disclosed herein.

In yet another aspect, the invention generally relates to a composition comprising a fusion protein disclosed herein.

In yet another aspect, the invention generally relates to a pharmaceutical composition comprising a therapeutically effective amount of a fusion protein disclosed herein.

In yet another aspect, the invention generally relates to a unit dosage form comprising a fusion protein disclosed herein.

In yet another aspect, the invention generally relates to a unit dosage form comprising a pharmaceutical composition disclosed herein.

In certain embodiments, the pharmaceutical composition further comprises a second therapeutic agent. In certain embodiments, the second therapeutic agent is an antiviral agent. In certain embodiments, the second therapeutic agent is an anti-inflammatory agent.

The composition or unit dosage form of the invention may be suitable for intravenous, intramuscular, subcutaneous, and/or inhaled administration.

In yet another aspect, the invention generally relates to a method for preparing the fusion protein, the method comprises: culturing a host cell comprising an expression vector comprising a nucleic acid encoding the fusion protein disclosed herein; expressing the nucleic acid to fusion protein; and recovering the fusion protein from the host cell culture.

In yet another aspect, the invention generally relates to a method for treating or reducing a coronavirus infection, or a related disease or condition, comprising administering to a subject in need thereof a therapeutically effective amount of a fusion protein disclosed herein, and a pharmaceutically acceptable excipient, carrier, or diluent.

In certain embodiments, the viral infection comprises infection of one or more of hCoV-229E, SARS-related coronaviruses, MERS-related coronaviruses.

In certain embodiments, the viral infection comprises infection of SARS-CoV-2.

In certain embodiments, the related disease or condition is pneumonia. In certain embodiments, the related disease or condition is ARDS. In certain embodiments, the related disease or condition is an inflammatory disorder. In certain embodiments, the related disease or condition is a cardiovascular disorder. In certain embodiments, the related disease or condition is a common cold or flu.

Any suitable route of administration may be selected, such as intravenous, intramuscular, subcutaneous, or inhaled administration.

In certain embodiments, the method further comprises administering a second therapeutic agent.

In certain embodiments, the second therapeutic agent is administered prior to, concomitant with or after the administration of the fusion protein.

In certain embodiments, the second therapeutic agent is an antiviral agent.

In certain embodiments, the second therapeutic is a nucleos(t)ide inhibitor or a protease inhibitor. In certain embodiments, the second therapeutic agent is a steroid. In certain embodiments, the second therapeutic agent is Remdesivir.

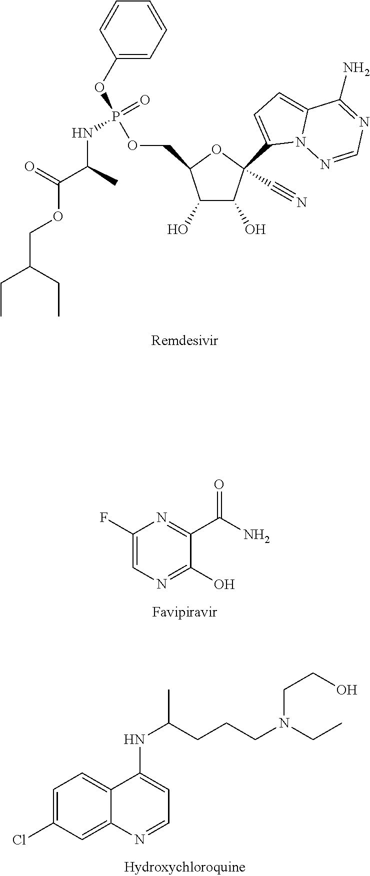 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1 ; NC(=O)c1nc(F)cnc1O ; CCN(CCO)CCCC(C)Nc1ccnc2cc(Cl)ccc12]

In certain embodiments, the antiviral second agent is selected from the group consisting of favipiravir, hydroxychloroquine, chloroquine, umifenovir, balapiravir, celgosivir, lovastatin, ribavirin, simeprevir, sofosbuvir, saquinavir, ritonavir, indinavir, nelfinavir, lopinavir-ritonavir, atazanavir, fosamprenavir, tipranavir, darunavir, darunavir+cobicistat, simeprevir, asunaprevir and vaniprevir.

In certain embodiments, the second therapeutic agent is a type I or type II interferon. In certain embodiments, the second therapeutic agent is selected from interferon alfa-2a (Roferon-A), interferon alfa-2b (Intron-A), interferon alfa-n3 (Alferon-N), peginterferon alfa-2b (PegIntron, Sylatron), interferon beta-1a (Avonex), interferon beta-1a (Rebif), interferon beta-1b (Betaseron), interferon beta-1b (Extavia), interferon gamma-1b (Actimmune), peginterferon alfa-2a (Pegasys ProClick), peginterferon alfa-2a and ribavirin (Peginterferon), peginterferon alfa-2b and ribavirin (PegIntron/Rebetol Combo Pack), peginterferon beta-1a (Plegridy), and interferon alfacon-1.

In yet another aspect, the invention generally relates to a method for inhibiting viral replication in cells, comprising administering to a subject in need thereof a therapeutically effective amount of a fusion protein comprising IFNT, or a fragment thereof, and the Fc portion of a human IgG comprising a hinge region, an IgG CH2 domain and an IgG CH3 domain, and a pharmaceutically acceptable excipient, carrier, or diluent.

In yet another aspect, the invention generally relates to use of a fusion protein comprising IFNT, or a fragment thereof, and the Fc portion of a human IgG comprising a hinge region, an IgG CH2 domain and an IgG CH3 domain, for treating a coronavirus infection, or a related disease or condition.

In yet another aspect, the invention generally relates to use of a fusion protein comprising IFNT, or a fragment thereof, and the Fc portion of a human IgG comprising a hinge region, an IgG CH2 domain and an IgG CH3 domain, in preparation of a medicament for treating a coronavirus infection, or a related disease or condition.

In certain embodiments, the coronavirus infection comprises infection of one or more of hCoV-229E, SARS-related coronaviruses, MERS-related coronaviruses.

In certain embodiments, the viral infection comprises infection of SARS-CoV-2.

In certain embodiments, the related disease or condition is one or more of pneumonia, ARDS, an inflammatory disorder, and a cardiovascular disorder.

In certain embodiments, IFNT Fc-fusion protein is administered at a dosage in the range from about 0.1 mg to about 200 mg (e.g., from about 0.1 mg to about 150 mg, from about 0.1 mg to about 100 mg, from about 0.1 mg to about 50 mg, from about 0.1 mg to about 10 mg, from about 0.1 mg to about 1 mg, from about 1 mg to about 200 mg, from about 10 mg to about 200 mg, from about 50 mg to about 200 mg, from about 100 mg to about 200 mg) per day.

In certain embodiments, administration of IFNT Fc-fusion protein is repeated for about 1 to about 30 days. In certain embodiments, a subject takes IFNT for about 3 to about 21 days (e.g., 3, 4, 5, 6, 7, 8, 9, 10, 11, 12, 13, 14, 15, 16, 17, 18, 19, 20, 21 days). In certain embodiments, a subject takes IFNT for about 7 to about 14 days (e.g., 7, 8, 9, 10, 11, 12, 13, 14 days).

The following examples are meant to be illustrative of the practice of the invention and not limiting in any way.

### EXAMPLES

The below Examples describe certain exemplary embodiments of compounds prepared according to the disclosed invention. It will be appreciated that the following general methods, and other methods known to one of ordinary skill in the art, can be applied to compounds and subclasses and species thereof, as disclosed herein.

### Example 1. Anti-SARS-CoV-2 Therapy

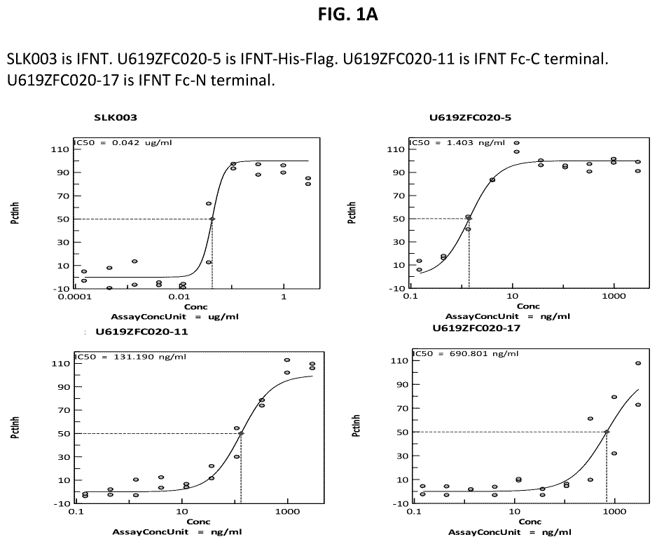

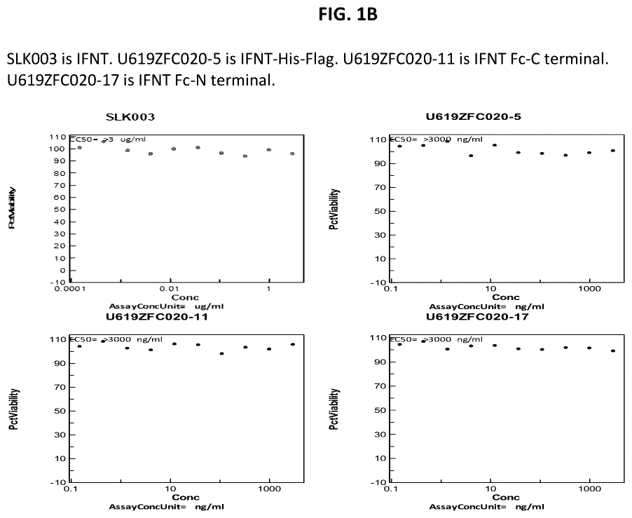

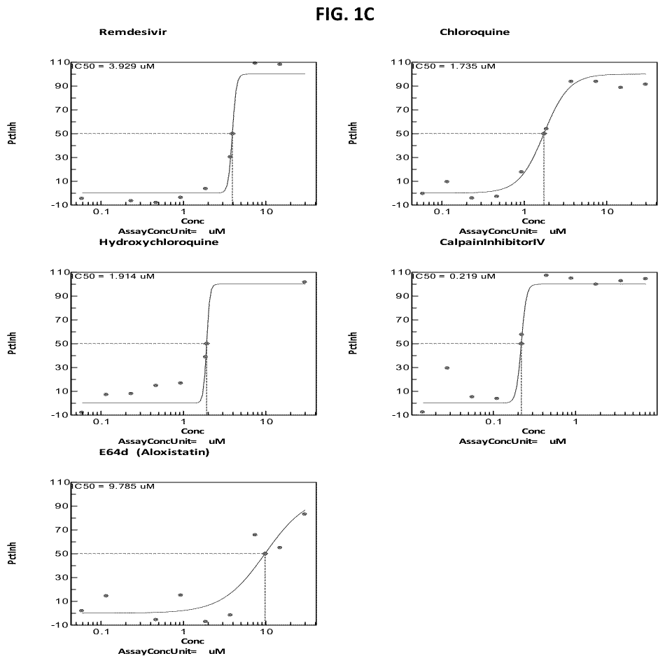

The anti-SARS-CoV-2 activity of IFNT and other reference compounds are shown in FIG. 1.

FIG. 1 A presents exemplary data of IFNT and Fc-fusion IFNT proteins-induced inhibition of SARS-CoV-2 in CPE assay. SARS-CoV-2 is a member of Coronaviridae family. The method of this CPE assay is described as follows.

Screening Strategy: We employ a cell-based assay measuring the cytopathic effect (CPE) of the virus infecting Vero E6 host cells. The CPE reduction assay is a popular and widely used assay format to screen for antiviral agents because of its ease of use in high throughput screening (HTS). (Maddox, et al. 2008 J. Assoc. Lab. Automation 2008; 13:168-73; Severson, et al. 2007 J Biomol Screen 12(1):33-40.) In this assay, host cells infected with virus die as a consequence of the viral infection and a simple and robust cell viability assay is the readout. The CPE reduction assay indirectly monitors the effect of antiviral agents acting through various molecular mechanisms by measuring the viability of host cells three days after inoculation with virus. Antiviral compounds are identified as those that protect the host cells from the cytopathic effect of the virus, thereby increasing viability.

Preparation of Assay Ready Plates: Compound stock solution supplied as 0.7 mg/ml (IFNT) and 1 mg/ml (SLK804) in PBS were transferred into an Echo® Qualified 384-Well Polypropylene Source Microplate (Labcyte P-05525). The compound was serially diluted 3-fold in PBS nine times. Using a Labcyte ECHO 550 acoustic liquid handling system a 127.5 nL aliquot of each diluted sample was dispensed into wells of a Coming 3764BC assay plate. This resulted in a 235-fold dilution of each sample in a final assay volume of 30 L to give the following final concentrations (μg/ml) in the assay:

| | | | | | | | | | |
|---|---|---|---|---|---|---|---|---|---|
| 3.0 | 1.0 | 0.333 | 0.111 | 0.0370 | 0.0123 | 0.00412 | 0.00137 | 0.00046 | 0.00015 |

Method for measuring antiviral effect of compounds: Vero E6 cells selected for expression of the SARS CoV receptor (ACE2; angiotensin-converting enzyme 2) were used for the CPE assay. (Severson, et al. 2007 J Biomol Screen 12(1):33-40.) Cells were grown in MEM/10% HI FBS and harvested in MEM/1% PSG supplemented 2% HI FBS. Cells were batch inoculated with SARS CoV-2 (USA_WA1/2020) at M.O.I. ˜ 0.002 which results in ˜5% cell viability 72 hours post infection. A 5ul aliquot of assay media was dispensed to all wells of the assay plates, then the plates were transported into the BSL-3. In the BSL-3 facility a 25 μL aliquot of virus inoculated cells (4000 Vero E6 cells/well) was added to each well in columns 3-24. The wells in columns 23-24 contain virus infected cells only (no compound treatment). A 25 μL aliquot of uninfected cells was added to columns 1-2 of the assay plates for the cell only (no virus) controls. After incubating plates at 37° C./5% CO₂ and 90% humidity for 72 hours, 30 L of Cell Titer-Glo (Promega) was added to each well. Luminescence was read using a BMG CLARIOstar plate reader following incubation at room temperature for 10 minutes to measure cell viability. Raw data from each test well was normalized to the average signal of non-infected cells (Avg Cells; 100% inhibition) and virus infected cells only (Avg Virus; 0% inhibition) to calculate % inhibition of CPE using the following formula: % inhibition CPE=100*(Test Cmpd−Avg Virus)/(Avg Cells−Avg Virus). Plates were sealed with a clear cover and surface decontaminated prior to luminescence reading.

Method for measuring cytotoxic effect of compounds: Compound cytotoxicity was assessed in a BSL-2 counter screen as follows: Host cells in media were added in 25 μl aliquots (4000 cells/well) to each well of assay plates prepared with test compound as above. Cells only (100% viability) and cells treated with hyamine at 100 μM final concentration (0% viability) served as the high and low signal controls, respectively, for cytotoxic effect in the assay. After incubating plates at 37° C./5% CO₂ and 90% humidity for 72 hours, plates were brought to room temperature and 30 μl Cell Titer-Glo (Promega) was added to each well. Luminescence was read using a BMG PHERAstar plate reader following incubation at room temperature for 10 minutes to measure cell viability.

Results are presented in Table 1. IFNT potently inhibits SARS-CoV-2 in CPE assay with IC50=2.1 nM, which is 1857 times more potent than remdesivir (IC50=3.9 μM), and 910 times more potent than Hydroxychloroquine (IC50=1.91 μM) in the same assay. Interestingly, the IC50 of IFNT-His-Flag is 0.06 nM, which is 35 times more potent than original IFNT. The IC50 of IFNT-Fc-C terminal is 1.38 nM, which is also lower than original IFNT. The IC50 of IFNT-Fc-N-terminal is 7.28 nM.

FIG. 1B presents exemplary cell viability data of IFNT and Fc-fusion IFNT proteins. Cytotoxicity evaluation was conducted in parallel with CPE assay. Cytotoxic effect of IFNT was also tested on host Vero E6 cells at the same ten concentrations used for the anti-viral assay in parallel. Cell viability was measured using Promega Cell Titer Glo. CC₅₀ values were calculated from a four-parameter logistic fit of the data.

FIG. 1C shows exemplary data of Remdesivir, chloroquine, hydroxychloroquine, aloxistatin, Calpain Inhibitor IV in the anti-SARS-CoV-2 CPE assay. The assays were performed as in FIG. 1A.

Table 1 shows the exemplary data of the anti-SARS-CoV-2 CPE assay.

TABLE 1
SARS-CoV2 CPE assay data

| | Antiviral | Cytotoxicity | Antiviral | Cytotoxicity | Selectivity Index |
|---|---|---|---|---|---|
| | IC₅₀ | CC₅₀ | IC₅₀ | CC₅₀ | (CC₅₀/IC₅₀) |
| Compound Name | (ng/ml) | (ng/ml) | (nM) | (nM) | (Based upon nM) |
| IFNT | 42 | >3000 | 2.11 | >150 | >71.45 |
| U619ZFC020-5 | 1.403 | >3000 | 0.06 | >126.47 | >2107.8 |
| (IFNT-His-Flag) | | | | | |
| U619ZFC020-11 | 131.190 | >3000 | 1.38 | >31.61 | >22.91 |
| (IFNT Fc-C terminal) | | | | | |
| U619ZFC020-17 | 690.801 | >3000 | 7.28 | >31.61 | >4.34 |
| (IFNT Fc-N terminal) | | | | | |

### Example 2. Anti-hCoV-229E Therapy

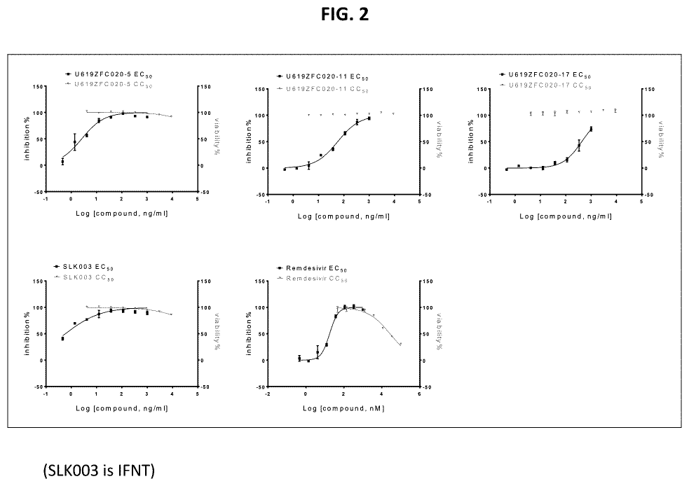

FIG. 2 present exemplary data of IFNT, IFNT-His-Flag (U619ZFC020-5), and two Fc-fusion IFNT proteins products (U619ZFC020-11, U619ZFC020-17) and Remdesivir inhibited hCoV229E in CPE assay. The method of this CPE assay is as follows: In 96-well plates, MRC5 cells were seeded at an appropriate density and cultured at 37° C. and 5% CO₂ overnight. Test samples were added into wells and the plates were incubated (200 TCID 50 hCoV-229E vs 20,000 MRC5 cells) at 37° C. and 5% CO₂ for 2 hours. Then medium in each well was replenished with medium containing serially diluted samples and virus. The resulting cultures were kept under the same conditions for additional 3 days until virus infection in the virus control displayed significant CPE. Cytotoxicity of the compounds was assessed under the same conditions, but without virus infection, in parallel. Cell viability was measured by CellTiter Glo following the manufacturer's manual. IC50 and CC₅₀ values were calculated with GraphPad Prism software.

Table 2 shows exemplary data of IFNT potently inhibited the hCoV229E with an IC50 of 0.03 nM, which is 500× more potent than Remdesivir's IC50 of 17.69 nM.

TABLE 2
HCoV-229E CPE assay data

| | Antiviral | Cytotoxicity | Antiviral | Cytotoxicity | Selectivity Index |
|---|---|---|---|---|---|
| | IC₅₀ | CC₅₀ | IC₅₀ | CC₅₀ | (CC₅₀/IC₅₀) |
| Compound Name | (ng/ml) | (ng/ml) | (nM) | (nM) | (Based upon nM) |
| IFNT | 0.58 | >9000 | 0.03 | >150 | >5000 |
| U619ZFC020-5 | 2.59 | >9000 | 0.11 | >126.47 | >1149.73 |
| (IFNT-His-Flag) | | | | | |
| U619ZFC020-11 | 56.80 | >9000 | 0.60 | >31.61 | >52.68 |
| (IFNT Fc-C terminal) | | | | | |
| U619ZFC020-17 | 423.90 | >9000 | 4.47 | >31.61 | >7.07 |
| (IFNT Fc-N terminal) | | | | | |
| Remdesivir | | | 17.69 | 26690 | 1508.76 |

Below Examples describe certain exemplary embodiments of compounds prepared according to the disclosed invention. It will be appreciated that the following general methods, and other methods known to one of ordinary skill in the art, can be applied to compounds and subclasses and species thereof, as disclosed herein.

### Example 3. IFNT Fc-Fusion Proteins

Exemplary Procedures to generate IFNT-His-Flag and IFNT Fc-fusion proteins are provided below.

### 1. Plasmid Preparation:

### General Procedure

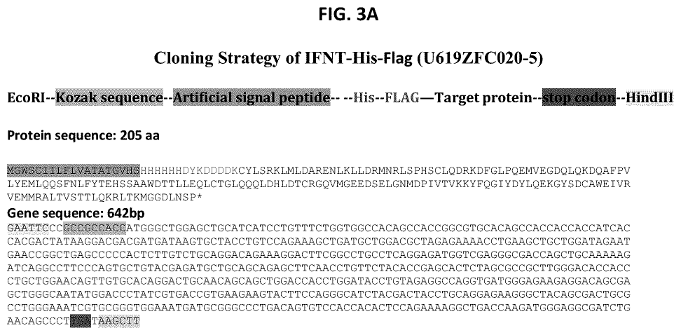

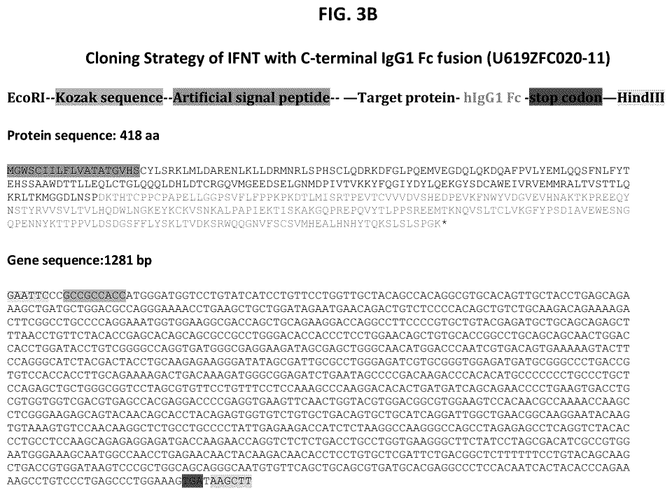

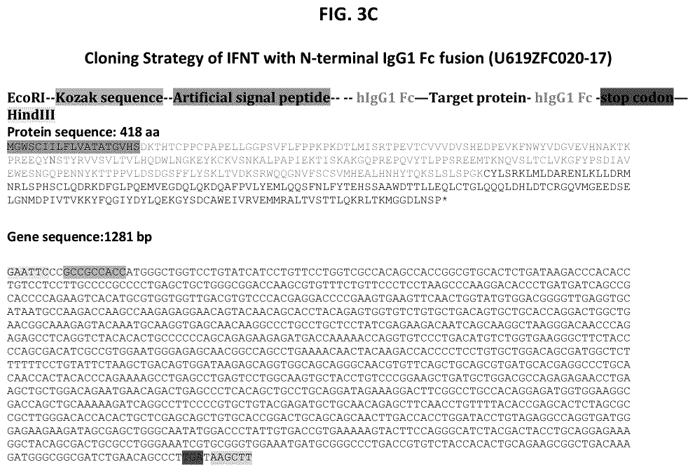

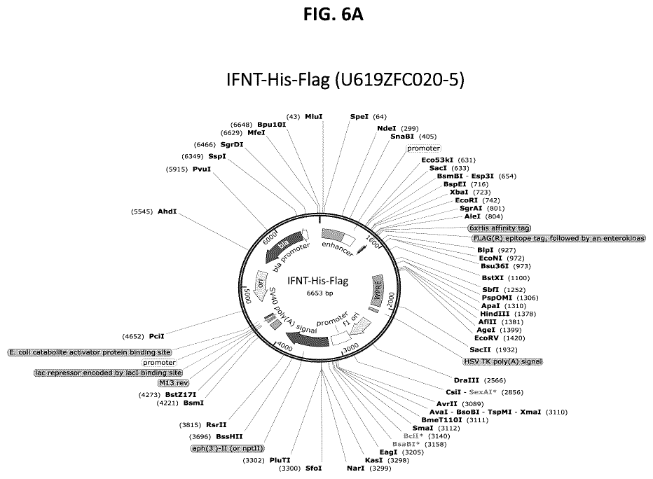

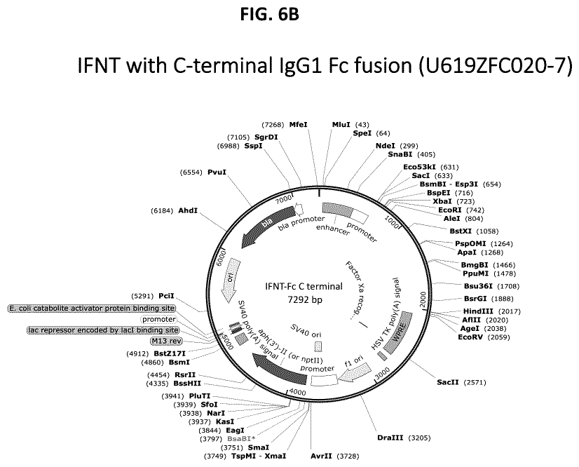

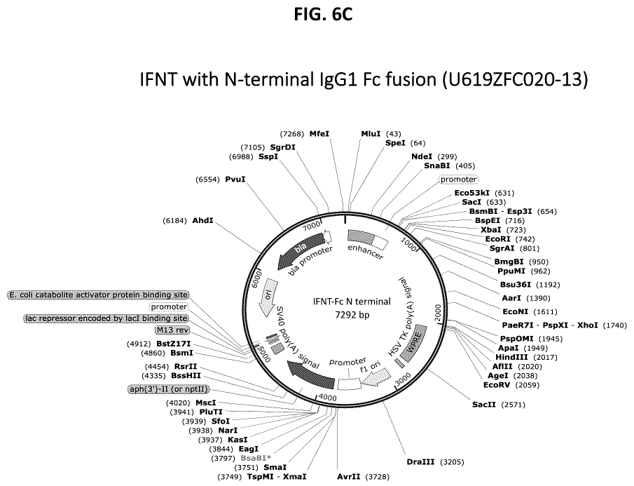

1) Target DNA sequence was designed, optimized and synthesized; the cloning sequences are in FIG. 3A-C. The plasmid maps are in FIG. 6A-C.
2) The complete sequence was sub-cloned into pcDNA3.4 vector.
3) Transfection grade plasmid was maxi-prepared for HD 293F cell expression.

### 2. Cell Culture and Transient Transfection:

1) HD 293F cells were maintained in Erlenmeyer Flasks (Coming Inc.) at 37° C. with 8% CO₂ on an orbital shaker (VWR Scientific).
2) One day before transfection, the cells were seeded at an appropriate density in Coming Erlenmeyer Flasks.
3) On the day of transfection, DNA and transfection reagent were mixed at an optimal ratio and then added into the flask with cells ready for transfection.
4) The recombinant plasmids encoding target protein was transiently co-transfected into suspension High-Density (HD) 293F cell cultures (GenScript proprietary HD transient expression system).
5) The cell culture supernatants collected on day 6 were used for purification.

### 3. Purification and Analysis:

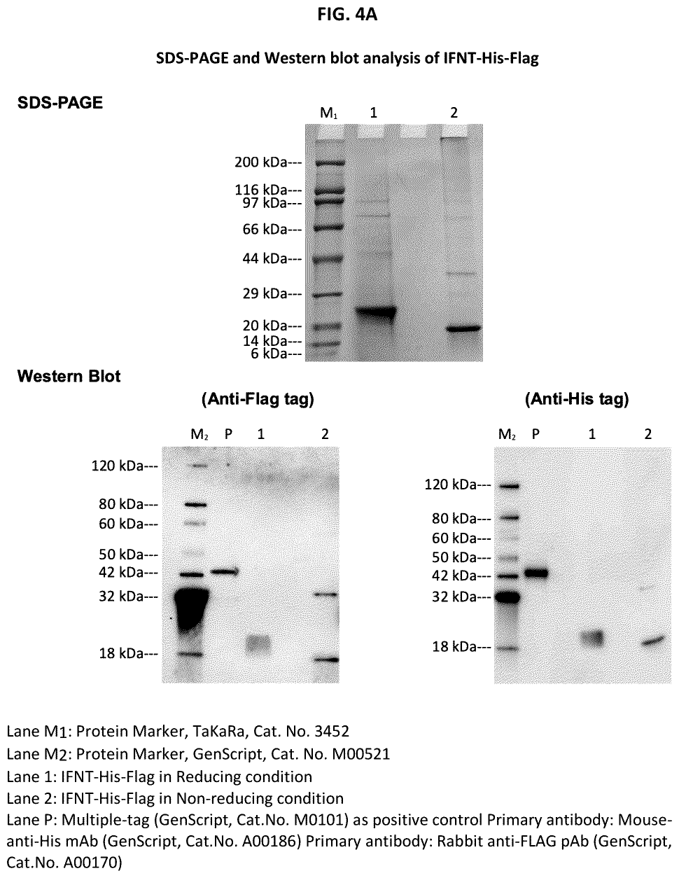

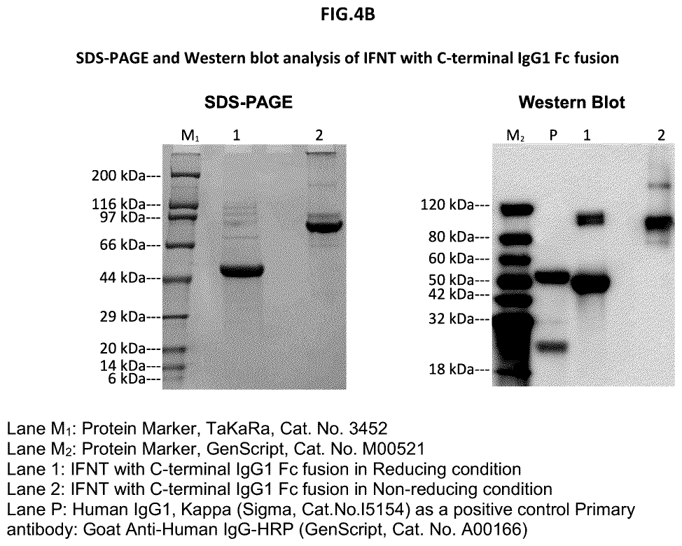

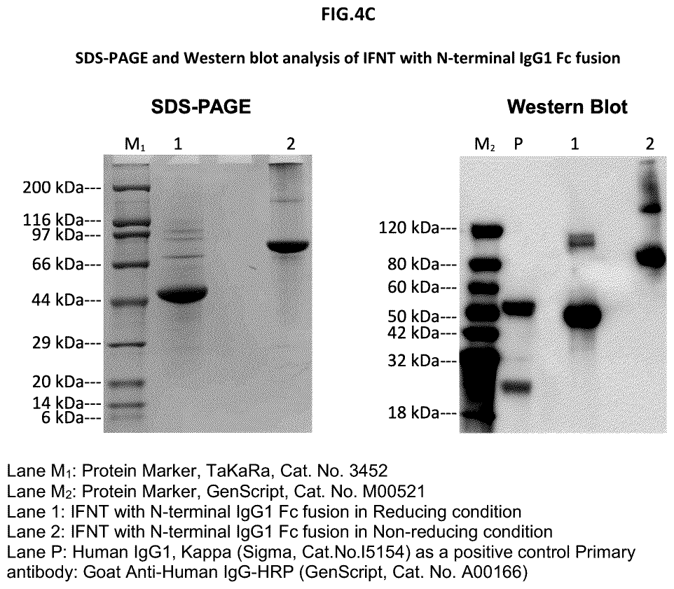

1) Cell culture broth was centrifuged and followed by filtration.
2) Filtered cell culture supernatant was loaded onto an affinity purification column at an appropriate flowrate.
3) After washing and elution with appropriate buffers, the eluted fractions were pooled and buffer exchanged to the final formulation buffer.
4) The purified protein was analyzed by SDS-PAGE, Western blot (FIG. 4A-C) analysis to determine the molecular weight and purity.
5) The concentration was determined by Bradford assay with BSA as a standard.

### 4. SDS-PAGE Analysis (FIG. 4A-C)

### Detailed Procedure:

1) Reducing and non-reducing loading buffer were added to protein sample respectively and the final concentration of protein was closed to 0.5 mg/ml. Then fully mix the mixture using MixPlus.
2) Heating the protein sample at 100° C. for 5-10 min (Only reducing condition)
3) Centrifuge protein samples (under reducing and non-reducing conditions) at 10000 rpm for 1 min, take the supernatant for SDS-PAGE analysis.
4) Fix the precast gel (GenScript, Cat. No. M42012) on electrophoretic apparatus, top up the inside groove with MOPS buffer.
5) Add 10 μl protein samples(under reducing and non-reducing conditions) in gel hole.
6) Run SDS-PAGE at 140V for 60 min, stop running when the bromophenol blue reached the bottom of separation gel and take the gel out.

The Reducing Loading buffer contains 300 mM Tris-HCl, 10% SDS, 30% Glycerol, 0.5% bromophenol blue, 250 mM DTT, pH 6.8. The Non-Reducing Loading buffer contains 300 mM Tris-HCl,10% SDS, 30% Glycerol, 0.5% bromophenol blue, pH 6.8 Gel: 4%˜20% gradient SDS-PAGE gel (GenScript Cat. No. M42012)

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term “consisting essentially of” does not exclude pharmacologically inactive or inert agents, e.g., pharmaceutically acceptable excipients, carriers or diluents. The term “consisting of” shall mean excluding trace elements of other ingredients and substantial method steps. Embodiments defined by each of these transition terms are within the scope of this invention.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A fusion protein comprising
interferon tau (IFNT), or a fragment thereof; and
the Fc portion of a human IgG comprising a hinge region, an IgG CH2 domain and an IgG CH3 domain.

2. The fusion protein of claim 1, wherein the human IgG is IgG1.

3. The fusion protein of claim 1, wherein the Fc portion of the human IgG is at the C terminal of IFNT.

4. The fusion protein of claim 1, wherein the Fc portion of the human IgG is at the N terminal of IFNT.

5. The fusion protein of claim 1, wherein the IFNT comprises a mammalian IFNT.

6. The fusion protein of claim 5, wherein the IFNT comprises a non-human mammalian IFNT.

7. The fusion protein of claim 1, wherein the IFNT comprises recombinant IFNT.

8. The fusion protein of claim 1, comprises an amino acid sequence that is at least 80% homologous with SEQ ID No. 1 or SEQ ID No. 2.

9. The fusion protein of claim 8, comprises an amino acid sequence having at least 80% homologous with SEQ ID NO. 1.

10. The fusion protein of claim 8, comprises an amino acid sequence having at least 80% homologous with SEQ ID NO. 2.

11. The fusion protein of claim 1, further comprising an IFNT with a His-Flag tag.

12. (canceled)

13. A purified fusion protein according to claim 1.

14. An isolated nucleic acid encoding the fusion protein of claim 1.

15. An expression vector comprising the nucleic acid of claim 14.

16. (canceled)

17. A host cell comprising the expression vector of claim 15.

18. (canceled)

19. A pharmaceutical composition comprising a therapeutically effective amount of the fusion protein of claim 1.

20. A unit dosage form comprising a fusion protein of claim 1.

21. (canceled)

22. The pharmaceutical composition of claim 21, further comprising a second therapeutic agent.

23. The pharmaceutical composition of claim 22, wherein the second therapeutic agent is an antiviral agent or anti-inflammatory agent.

24-26. (canceled)

27. A method for treating or reducing a coronavirus infection, or a related disease or condition, comprising administering to a subject in need thereof a therapeutically effective amount of a fusion protein of claim 1, and a pharmaceutically acceptable excipient, carrier, or diluent.

28-50. (canceled)
